(19) United States
(12) Patent Application Publication
(10) Pub. No.: US 2003/0215455 A1
(43) Pub. Date: Nov. 20, 2003

(54) Vaccine stabilizer and method of use

(76) Inventors: Bailey Reynolds, Nacogdoches, TX (US); Ryan S. Izard, Nacogdoches, TX (US)

Correspondence Address: ROBINSON & POST, L.L.P., North Texas Bank Tower, Suite 575, 12900 Preston Road, LB-29, Dallas, TX, 75230-1329, US

(21) Appl. No.: 10/145,444

(22) Filed: May 14, 2002

(51) Int. Cl. A61K 39/00; A61K 31/70

(52) U.S. Cl. 424/184.1; 514/23

(57) ABSTRACT

A vaccine stabilizer for adjusting water quality that adversely effects the life of a vaccine to be administered to livestock. The stabilizer is composed of a reducing agent to neutralize oxidizing water sanitizers of at least about 0.0002 percent by weight and a coloring agent to provide a visual reference for aiding in determining the administration of the vaccine to the livestock of from about 0.00 to about 99.9998 percent by weight.

### BACKGROUND OF THE INVENTION

This invention relates to a vaccine stabilizer for maintaining the infectivity of live viral and bacterial vaccines for animals and methods of using of a vaccine stabilizer.

It is well known that a large variety of infectious organisms negatively affects the health, well-being, and productivity of farm animals, commonly called livestock. To fight these infectious organisms, it is common for the animal caretakers to inject, spray, provide in drinking water or otherwise administer vaccines to such livestock. Commonly, these vaccines are attenuated or avirulent live infections strains of the viral or bacterial antigens. When the vaccines are kept viable, they confer increased disease resistance to the animal and improve the animal's health and productivity.

Over the past several years, the farm size has been increasing. To cope with the increased farm size, animal caretakers now require mass vaccination of animals via spray and drinking water over individual inoculation by injection. This mass administration of vaccine by aerosol spray or drinking water benefits those producing the animals by reducing labor and eliminating the injection site injuries and broken needle residue that threatens the quality and safety of meat products.

The attenuated or avirulent live organisms used by the animal caretakers are sensitive to changes in their environment and degrade when exposed to suboptimal conditions. Thus, vaccine stabilizers are used as agents added to liquid, frozen, or lyophilized vaccines to extend the vaccine infectivity and maintain effectiveness. These prior art stabilizers are specifically formulated for the makeup and conditions experienced by each vaccine. Normally, the prior art stabilizers are incorporated with the vaccine in the original container and stabilize the vaccine throughout manufacturing, storage and warming. They impart greater shelf life to the vaccine until the vaccine is readied for injection. While stabilizers in a vaccine are effective at extending a vaccine's infectivity throughout storage until injection, the practice of mass administration by spray or drinking water exposes the vaccine to increased hazards that can reduce its effectiveness.

One problem occurs when the temperature of a vaccine raises above the optimum storage condition. As the temperature raises the potency of the vaccine erodes. This temperature sensitivity necessitates refrigerated storage and careful warming for the vaccine to remain effective. Because a lack of adequate refrigeration exists at many animal confinements, vaccines are frequently stored in centralized locations. After the vaccine is removed from cold storage and transported to the outlying facilities, the temperature increases and the vaccine begins to decay as it is removed from its original container and rehydrated. This temperature increase continues while the vaccine is added to a sufficient volume of water to be sprayed on the animals, misted in their nostrils, or slowly and proportionately metered into their drinking water and continues while mixing and administering the vaccine.

Also, the vaccine potency is adversely affected by the water or diluent used as the delivery vehicle to mass administer the livestock on the farms. The water or diluent typically contains oxidizing sanitizers such as chlorine, peroxide, bromine, and the like used in municipalities. While these sanitizers disinfect the water of common pathogenic organisms, they also kill the infectious agents present in live vaccines. The result is a complete loss of the vaccine's potency and failure to protect the animals from subsequent infections.

Further, there are other factors that can adversely effect the vaccine's viability. These factors include pH excursions beyond optimum limits for the vaccine, and organic oxidizers, which include nitrites and less commonly sulfites and chloramines.

Vaccine manufacturers have recognized the perils of subjecting their vaccine to the inhospitable conditions inherent to farm water supplies in the past. The manufactures have recommended that the vaccine user purchase and transport distilled or deionized bottled water to be used as a vaccine diluent or carrier for sprayed vaccine. However, when poultry production units vaccinate 15,000-20,000 birds at a single confinement building with a vaccination crew moving to 12-16 such facilities each working day, it becomes impractical to obtain and transport the necessary large volumes of bottled distilled or deionized water for sprayed vaccines and impossible for vaccines delivered via the farm's drinking water system. On another occasion, vaccine manufacturers have recommended that animal caretakers mix large quantities of powdered milk with water supplies to aid in reducing the free chlorine in the water prior to mixing the vaccine. However, the large volumes of powdered milk required to effectively reduce free chlorine are impractical. Also, the powdered milk does not fully dissolve in the cold water and this undissolved milk powder collects in vaccine delivery systems, and clogs the spray nozzles and orifices of drinking water dispensers. The clogged vaccinating equipment is thereby prevented from functioning properly and fails to vaccinate the animals uniformly. Thereby, failing to confer immunity to the entire group.

A need exists for a novel vaccine stabilizer that will be effective after the vaccine is opened and throughout the administration to the animals in sanitized tap water or well water spray or drinking water. Such a novel stabilizer will prolong the infectivity of both live viral and bacterial vaccine by reducing negative water quality factors that limit the life of the vaccine. The stabilizer will permit farms or animal caretakers to use their own integral water supply as a functional delivery vehicle for the vaccine and ultimately afford the animals greater protection from disease.

### BRIEF SUMMARY OF THE INVENTION

In accordance with the present invention, there is provided a vaccine stabilizer for adjusting water quality that adversely effects the life of a vaccine to be administered to livestock. The stabilizer comprises a reducing agent to neutralize oxidizing water sanitizers of at least about 0.0002 percent by weight. A buffer is used to adjust the pH of from about 0.00 to about 20 percent by weight. A thermal stabilizer for the vaccine of from about 0.00 to about 75 percent by weight is included. A coloring agent to provide a visual reference for aiding in determining the administration of the vaccine to the livestock of from about 0.00 to about 3.5 percent by weight is included. A sugar for an energy source for the vaccine of from about 0.00 to about 85 percent by weight is included. Water of from about 0.00 to about 99.9998 percent by weight is included.

Further, in accordance with the present invention, there is provided a method of treating livestock with a vaccine. A quantity of water for treating livestock with the vaccine is provided. A reducing agent selected to neutralize oxidizing water sanitizers is mixed in the water. The vaccine is mixed in the water after the reducing agent has neutralized the water sanitzers. The livestock is then treated with the vaccine and water mixture.

Further, in accordance with the present invention, there is provided a method of treating livestock with a vaccine. Water is transported to a location for treating livestock with a vaccine. A reducing agent that neutralizes oxidizing water sanitizers being transported in the water while not adversely effecting the vaccine is selected. The selected reducing agent is added to the water before reaching the location. The vaccine is mixed in the water after the reducing agent is added to the water and before the water reaches the location.

### DETAILED DESCRIPTION OF THE INVENTION

The vaccine stabilizer of this invention comprises agents that confer extended stability to a live viral or bacterial vaccine being carried by water.

The stabilizer can be either a dry or a liquid form that is suitable for addition to tap or well water or similar diluent prior to the introduction of the vaccine. The state of the stabilizer can be liquid or dry depending on the user's choice.

A reducing agent is used in the stabilizer to neutralize oxidizing sanitizers or contaminants present in the farm water. The reducing agent is selected in an amount appropriate to neutralize the oxidizing sanitizers or contaminants present in the farm water that is to be used as the conveyance vehicle for the vaccine. Although the amount of reducing agent used in the vaccine stabilizer of the present invention is at least about 0.0002 percent by weight, it is more preferred that at least about 0.144 percent by weight be used and most preferred that at least about 0.1952 percent by weight be used. Examples of the oxidizing sanitizer and other contaminants are chlorine, peroxide, bromine, fluorine, ozone, permanganate, chromic acid, chloramines, and nitrites. Preferably, the reducing agent is at least one chemical selected from, but not limited to, the group consisting of sodium thiosulfate, sodium metabisulfite, sodium bisulfite, sodium sulfite, sulfur dioxide, ammonium bisulfite, and ammonium thiosulfate. Most preferred is sodium thiosulfate because it is effective over a range of pH levels, and is generally recognized as a safe food additive in Code of Federal Regulations (CFR) 21 582.6807, as being non-toxic. Also, it is non-corrosive, highly water-soluble, and has a pH near neutrality in solutions of water.

When desired, the stabilizer includes a biologically acceptable sugar to serve as an energy source for stabilized live bacterial vaccine or cell culture media. The sugar serves as a readily water-soluble carrier for the reducing agent. The sugar is selected from the group including, but not limited to, glucose, dextrose, lactose, sucrose, mannose, and fructose. Although the amount of the sugar used may be from about 0.00 to about 99.9998 percent by weight, it is preferred that about 0.00 to about 85.00 percent by weight be used.

Also, when desired, buffering agents are used to keep the pH of the stabilized vaccine preparation generally in a range of about 6-7, which is appropriate for a spectrum of viral and bacterial antigens. The pH is variously adjusted by use of the buffering agents to neutralize digestive acids or balance the water to a physiological pH to accommodate cell cultures when necessary. The buffering agents preferably used with the stabilizer are phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. Although the amount of buffer used in the vaccine stabilizer is from about 0.00 to about 99.9998 percent by weight, it is preferred that from about 0.00 to about 20.0 percent by weight be used.

When desired, a protein source is used to improve thermal stability. One such protein source known to improve thermal stability in lyophilized viral vaccine preparations is disclosed by Volkin, et al. in U.S. Pat. No. 6,290,967. Other water-soluble proteins that are acceptable thermal stabilizers are sodium caseinate, calcium caseinate, isolated soy protein, serum albumin, egg albumin, and the like. Also, d- or l-lysine, d- or l-arginine, or other similar compounds bearing two amino or imine groups separated by a spacer moiety is disclosed by Dorval, et al. in U.S. Pat. No. 5,618,539 to improve the thermal stability of certain non-lyophilized injectable vaccines. Although used in injectable vaccine stabilizers, these agents have not been used as a stabilizer for sprayed or oral vaccines administered in tap water. Although the amount of thermal stablizer that may be used in the vaccine stabilizer is from about 0.00 to about 99.9998 percent by weight, it is preferred that about 0.00 to about 75.00 percent be used.

Another ingredient in the stabilizer is a water-soluble FD&C food coloring approved by the FDA. The coloring provides visual verification to the animal caretaker that the stabilizer has been added to the water, and the stabilized water solution is prepared to receive the vaccine. Additionally, the colorant remains in the vaccine spray or drinking water to mark the feathers, skin, hair, wool, lips or tongues of the animals that have been sprayed or that have consumed the water. Such marking aids the health management of the animals by serving as a visual reference to the caretaker for positively identifying the vaccinated and non-vaccinated animals. Although the amount of coloring agent used in the vaccine stabilizer of the present invention is from about 0.00 to about 3.5 percent by weight, it is preferred that at least about 0.002 percent by weight be used, more preferred that at least about 0.0064 percent by weight be used, and most preferred that at least about 0.1125 percent by weight be used.

This invention contemplates both dry and liquid physical states of the stabilizer. The dry embodiments contain ingredients appropriately selected, blended, and stored in a dry state to be dissolved in the vaccine vehicle when use is eminent. The liquid embodiments employ the use of less concentrated liquid stabilizer formulations that contain a physiologically acceptable liquid diluent and carrier, preferably water. On occasion, the skilled artisan may deem it more suitable to his or her purpose to produce the stabilizer as a liquid preparation instead of a dry mixture. Such occasions would likely arise when the concentration and handling characteristics of a diluted liquid concentrate would lead to better measuring and mixing in the water vehicle. The dry state would be employed when higher stabilizer concentrations are desired.

Animal caretakers can add the stabilizing mixture directly to ordinary tap water or farm water, which serves as the delivery vehicle in mass vaccine administrations. The stabilizer neutralizes harmful compounds in the water before those compounds act to decay the vaccine's potency. When desired, the stabilizer serves as a source of energy for certain vaccines, buffers the tap water against pH excursions, imparts improved thermal stability to the vaccine during vaccination, and marks the stabilized water and vaccinated animals for visual verification.

In the animal drinking system, a dry stabilizer composition is dissolved in water to form a “stock solution”, which is a premix of tap or farm well water, stabilizer ingredients, and an appropriate amount of vaccine doses for the animal group. The stabilizer is added first to neutralize the oxidizing sanitizers and contaminants, and makes the stock solution hospitable to the vaccine. The required doses of vaccine are then added to the stock solution, and the stock solution is further diluted in the animals' drinking water by a variety of means available to the caretaker. Typically, caretakers use a proportional injector device set to deliver 1 fluid ounce of stabilized vaccine in stock solution to each gallon (a concentration of 0.78%) of drinking water. The stabilizer present in the stock solution ensures that oxidizing sanitizers and contaminants in the greater volume of drinking water are also neutralized, rescuing the vaccine from potential decay. The animals drink the stabilized vaccine-laden water until all doses are consumed and all animals are vaccinated.

### EXAMPLE 1

A dry mix of 15 grams is made by mixing together 2.5 grams (16.67 percent by weight) of sodium thiosulfate as the reducing agent, 21 mg (0.14 percent by weight) sodium bicarbonate as the buffer, 21 mg (0.14 percent by weight) dried whey as the thermal stabilizer, 225 mg (1.50 percent by weight) FD&C Blue #1 as the coloring agent, 12.233 grams (81.55 percent by weight) dextrose as the sugar, and 0.00 grams (0.00 percent by weight) water or other diluent.

These 15 grams of dry mix are then added to water to form a 1 liter liquid premix or concentrated stock solution. This concentrated stock solution is then introduced into the drinking water at a rate of about 1 fluid ounce of concentrate per gallon of drinking water (0.78%).

### EXAMPLE 2

A dry mix of 200 grams is made by mixing together 2.5 grams (1.25 percent by weight) of sodium thiosulfate as the reducing agent, 36 grams (18.00 percent by weight) sodium phosphate and 25 grams (1 percent by weight) sodium glutamate as the buffer, 146 grams (73.00 percent by weight) L-Lysine as the thermal stabilizer, 225 mg (0.1125 percent by weight) FD&C Blue #1 as the coloring agent, 13.275 grams (6.6375 percent by weight) sucrose as the sugar, and 0.00 grams (0.00 percent by weight) water or other diluent.

These 200 grams of dry mix are then added to water to form a 1 liter liquid premix or concentrated stock solution. This concentrated stock solution is then introduced into the drinking water at a rate of about 1 fluid ounce of concentrate per gallon of drinking water (0.78%).

### EXAMPLE 3

A dry stabilizer that may be used to protect and extend the potency of vaccine administered through a drinking water supply containing 4 ppm chlorine. 200 grams of dry mix is made by mixing together 288 mg (0.144 percent by weight) of sodium thiosulfate as the reducing agent, 36 grams (18.00 percent by weight) sodium phosphate and 2 grams (1 percent by weight) sodium glutamate as the buffer, 146 grams (73.00 percent by weight) L-Lysine as the thermal stabilizer, 225 mg (0.1125 percent by weight) FD&C Blue #1 as the coloring agent, 15.487 grams (7.7435 percent by weight) sucrose as the sugar, and 0.00 grams (0.00 percent by weight) water or other diluent.

These 200 grams of dry mix are then added to water to form a 1 liter liquid premix or concentrated stock solution. This concentrated stock solution is then introduced into the drinking water at a rate of about 1 fluid ounce of concentrate per gallon of drinking water (0.78%).

### EXAMPLE 4

A dry stabilizer that may be used to protect and extend the potency of vaccine administered through a drinking water supply containing 8 ppm chlorine. 200 grams of dry mix is made by mixing together 576 mg (0.288 percent by weight) of sodium thiosulfate as the reducing agent, 36 grams (18.00 percent by weight) sodium phosphate and 2 grams (1 percent by weight) sodium glutamate as the buffer, 146 grams (73.00 percent by weight) L-Lysine as the thermal stabilizer, 225 mg (0.1125 percent by weight) FD&C Blue #1 as the coloring agent, 15.199 grams (7.5995 percent by weight) sucrose as the sugar, and 0.00 grams (0.00 percent by weight) water or other diluent.

These 200 grams of dry mix are then added to water to form a 1 liter liquid premix or concentrated stock solution. This concentrated stock solution is then introduced into the drinking water at a rate of about 1 fluid ounce of concentrate per gallon of drinking water (0.78%).

When used in a spray solution, the liquid stabilizer composition set forth in the following examples are combined with tap water or farm well water to form a “spray solution”. The stabilizer neutralizes the oxidizing sanitizers and contaminants, and makes the spray solution hospitable to the vaccine. The required doses of vaccine are then added to the spray solution, and the animals are sprayed with the stabilized vaccine-laden water for a length of time that varies with the number of animals until all are vaccinated. In this instance, excess reducing agent is not necessary because the caretaker is not further diluting the spray solution in a greater volume of water that must also be stabilized.

### EXAMPLE 5

When administered in a sprayed solution, this vaccine stabilizer may be added directly to the water being sprayed or mixed with a diluent, such as water, for addition to the water being sprayed. A dry mix of 64 milligrams grams is made by mixing together 61 mg (95.3125 percent by weight) of sodium thiosulfate as the reducing agent, 0.5 mg (0.78125 percent by weight) sodium bicarbonate as the buffer, 0.5 mg (0.78125 percent by weight) Sorbitol as the thermal stabilizer, 2 mg (3.125 percent by weight) FD&C Red #40 as the coloring agent, 0.00 grams (0.00 percent by weight) sugar, and 0.00 grams (0.00 percent by weight) water or other diluent.

When mixed with a diluent, the solution forms a 31.25 ml liquid premix or concentrated stock solution and this amount of premix is then introduced into each liter of spray water or is introduced at a rate of about 4 fluid ounces for each gallon of water being sprayed.

### EXAMPLE 6

When administered in a sprayed solution, this vaccine stabilizer may be added directly to the water being sprayed or mixed with a diluent, such as water, that is then added to the water being sprayed. A dry mix of 300 grams is made by mixing together 61 mg (0.02033 percent by weight) of sodium thiosulfate as the reducing agent, 36 grams (12 percent by weight) sodium phosphate and 2 grams (0.6666 percent by weight) sodium glutamate as the buffer, 146 grams (48.6666 percent by weight) L-Lysine and 40 grams (13.3333 percent by weight) sorbitol as the thermal stabilizer, 2 mg (0.00066 percent by weight) FD&C Red #40 as the coloring agent, 75.937 grams (25.3123 percent by weight) sucrose as the sugar, and 0.00 grams (0.00 percent by weight water or other diluent.

When mixed with a diluent, the solution forms a 1 liter liquid stabilizer solution that is used as a spray when mixed with the vaccine.

### EXAMPLE 7

When administered in a sprayed solution, this vaccine stabilizer may be used by adding the dry mix directly to the water for spraying. This mix is prepared for a spray solution with water containing 4 ppm chlorine. A dry mix of 300 grams may be made by mixing together 2.22 mg (0.00074 percent by weight) of sodium thiosulfate as the reducing agent, 36 grams (12 percent by weight) sodium phosphate and 2 grams (0.6666 percent by weight) sodium glutamate as the buffer, 146 grams (48.6666 percent by weight) L-Lysine and 40 grams (13.3333 percent by weight) as the thermal stabilizer, 2 mg (0.00066 percent by weight) FD&C Red #40 as the coloring agent, 75.99578 grams (25.3319 percent by weight) sucrose as the sugar, and 0.00 grams (0.00 percent by weight) water or other diluent.

When mixed with water containing 4 ppm chlorine 1 liter spray solution is prepared.

### EXAMPLE 8

When administered by a sprayed solution, this vaccine stabilizer may be used by adding the dry mix directly to the water for spraying. This mix is prepared for a spray solution with water containing 8 ppm chlorine. A dry mix of 300 grams may be made by mixing together 4.45 mg (0.00148 percent by weight) of sodium thiosulfate as the reducing agent, 36 grams (12 percent by weight) sodium phosphate and 2 grams (0.6666 percent by weight) as the buffer, 146 grams (48.6666 percent by weight) L-Lysine and 40 grams (13.3333 percent by weight) as the thermal stabilizer, 2 mg (0.00066 percent by weight) FD&C Red #40 as the coloring agent, 75.99355 grams (25.3312 percent by weight) sucrose as the sugar, and 0.00 grams (0.00 percent by weight) water or other diluent.

When mixed with water containing 8 ppm chlorine, a 1 liter spray solution is prepared.

Evaluating the effectiveness of the reducing agents is most easily performed by testing the stabilized water for chlorine content. In the lab, a spectrophotometer such as a Hach model DR/2010 will read the chlorine content of a 10 ml sample after the addition of DPD reagent and correction with a blank standard sample.

Alternatively, chlorine may be measured in the field using a Hach Chlorine Test Kit to treat the test sample with a DPD reagent and then compare the color of the sample to a color comparison card while correcting for the background color of a blank standard sample.

Testing the extended stability of a vaccine in the presence of the stabilizer involves use of the biological method employed by vaccine manufacturers and described in Title 9, CFR 113.327, among others, wherein the mixed vaccine test sample is injected into fertile pathogen-free eggs. Vaccine that has been effectively stabilized is therefore potent and infective, and will produce deformities of the embryos. Vaccine that has not been effectively stabilized will not be fully infective and will produce fewer lesions than a more functional vaccine. This method is used to compare stabilized and unstabilized vaccines to each other, and evaluate the potency retention of a vaccine held for varying lengths of time.

Experiments were conducted to study the stabilizing properties of the liquid stabilizer composition. The composition of the stabilizer included a reducing agent of 7.8 grams/liter of Sodium Thiosulfate Pentahydrate; a coloring agent of 0.3 grams/liter of Disodium salt of ethyl [4-[p-[ethyl (m-sulfobenzyl) amino]-alpha-(o-sulfophenyl) benzylidene]-2,5-cyclohexadiene-1-ylidene], -sulfobenzyl, psulfobenzyl, and o-sulfobenzyl ammonium; and water.

For the three experiments, a common method of titrating the live virus concentration was used. Titration of live virus concentration was conducted with specific-pathogen-free embryonated eggs using the method commonly employed by vaccine manufacturers. The method is described in Title 9, CFR 113.327. Briefly, 0.1 ml of 10-fold serial dilutions of vaccine virus was inoculated in the allantoic cavity of groups of six embryos, 9- to 11-days of age. This dose was selected as one previously determined to provide 10^{4.4} embryo infective doses (EID₅₀) per 0.1 ml of administered vaccine volume. Embryo deaths occurring during the first 24 hours after inoculation were disregarded. After 6 or 7 days incubation, surviving embryos were examined for signs of infection, to include, stunting, curling, and clubbing. A satisfactory titer was obtained when at least four embryos survived in each dilution, one dilution produced 50 to 100 percent positives, and one dilution, 0 to 50 percent positives. The method of Reed and Muench was used to calculate EID₅₀ per 0.1 ml of administered vaccine volume. All titrations were replicated.

In order to ensure that the stabilizer did not pose a hazard to the relatively fragile vaccine virus, the effect of the stabilizer on the vaccine was compared to the effect of distilled water alone. The lyophilized vaccine was reconstituted in distilled water at the rate of 1000 doses per 100 ml (1 dose/0.1 ml) and then divided equally into two vials. To one of the vials, stabilizer was added at the rate of 0.8 ml per 100 ml. After 30 and 120 minutes, titrations of vaccine in each of the two vessels, respectively, were conducted.

The titer, or concentration, of the virus rehydrated in distilled water near neutral pH at room temperature was determined to be 10^{4.4} EID₅₀/0.1 ml at 30 minutes, deteriorating 20% to 10^{4.3} EID₅₀/0.1 ml at 120 minutes. The corresponding titers in stabilized water were 10^{4.5} EID₅₀/0.1 ml at both time intervals, an increase of 20% after 30 minutes and 50% after 2 hours. This result indicated that the stabilizer was at least not injurious to the vaccine, and at best improved the stability of the vaccine throughout the times tested.

The capacity of the stabilizer to protect the vaccine against the detrimental effect of chlorinated water was evaluated. The experiment compared the viability of vaccine virus rehydrated in chlorinated distilled water containing stabilizer to that of vaccine virus rehydrated in chlorinated distilled water alone. The available free chlorine was adjusted to 4 ppm, a level in the higher range of what would typically be encountered in municipal or rural cooperative water systems. The methodology of the second experiment was the same as that of the first except for the addition of sodium hypochlorite to the water used for rehydration.

As expected, chlorine at 4 ppm significantly degraded virus titer to 10^{3.7} EID₅₀/0.1 ml and 103.9 EID₅₀/0.1 ml at 30 and 120 minutes, respectively. In contrast, addition of stabilizer to the rehydrated virus prior to the introduction of chlorine prevented the virus degradation at both the 30- and 120-minute intervals. The potency decay in vaccine without stabilizer amounted to 75% at 30 minutes and 68% at 2 hours. Vaccine held in the presence of stabilizer for 2 hours increased titer by 25% over that held for 30 minutes (Table 3 and FIG. 2).

The purpose and methodology of the following test were identical to the above, except that the level of available chlorine was adjusted to 8 ppm. This level is over twice that typically found in municipal or rural cooperative water systems, but represents the potential over-chlorination that might occur on farms that chlorinate their own well water.

As was observed for the addition of chlorine at 4 ppm, chlorine at 8 ppm significantly degraded virus titer to 10^{3.7} EID₅₀/0.1 ml and 10^{3.9} EID₅₀/0.1 ml at 30 and 120 minutes, respectively. In contrast, addition of stabilizer to the rehydrated virus prior to the introduction of chlorine prevented the virus degradation at both the 30- and 120 minute intervals. The potency decay in vaccine without stabilizer amounted to a loss of 80% at 30 minutes and 88% at 2 hours. Vaccine held in the presence of stabilizer for 2 hours increased titer by 20% over that stabilized for only 30 minutes.

The results of the test show that the vaccine held in the presence of stabilizer for 120 minutes maintained at least the same titer as vaccine held for 30 minutes, and exceeded it in cases where chlorinated water was used. Thus, the stabilizer preparation is safe to the vaccine itself, and is capable of rescuing the fragile virus vaccine by defeating the harsh conditions imposed by a chlorinated water diluent.

The invention having been described, what is claimed is:

1. A vaccine stabilizer for adjusting water quality that adversely effects the life of a vaccine to be administered to livestock, comprising:
a reducing agent to neutralize oxidizing water sanitizers of at least about 0.0002 percent by weight and
a coloring agent to provide a visual reference for aiding in determining the administration of the vaccine to the livestock of from about 0.00 to about 99.9998 percent by weight.

2. The vaccine stabilizer set forth in claim 1, further including:
said reducing agent being at least about 0.144 percent by weight.

3. The vaccine stabilizer set forth in claim 2, further including:
said reducing agent being at least about 0.1952 percent by weight.

4. The vaccine stabilizer set forth in claim 1, further including:
said reducing agent being one chemical selected from the group consisting essentially of sodium thiosulfate, sodium metabisulfite, sodium bisulfite, sodium sulfite, ammonium bisulfite and ammonium thiosulfate.

5. The vaccine stabilizer set forth in claim 1, further including:
said coloring agent being at least 0.0002 percent by weight.

6. The vaccine stabilizer set forth in claim 5, further including:
said coloring agent being at least 0.0064 percent by weight.

7. The vaccine stabilizer set forth in claim 6, further including:
said coloring agent being at least 0.1125 percent by weight.

8. The vaccine stabilizer set forth in claim 1, further including:
said coloring agent being at least one chemical selected from the group consisting of a water soluble FD&C compliant food color.

9. The vaccine stabilizer set forth in claim 1, further including:
a buffer to adjust pH of from about 0.00 to about 99.9998 percent by weight.

10. The vaccine stabilizer set forth in claim 9, further including:
said buffer being from about 0.00 to about 20.00 percent by weight.

11. The vaccine stabilizer set forth in claim 9, further including:
said buffer being at least one chemical selected from the group consisting of phosphates, carboxylates and bicarbonates.

12. The vaccine stabilizer set forth in claim 11, further including:
said buffer being at least one chemical selected from the group consisting of sodium phosphate, potassium phosphate, sodium citrate, calcium lactate, sodium succinate, sodium glutamate, sodium bicarbonate, and potassium bicarbonate.

13. The vaccine stabilizer set forth in claim 1, further including:
a thermal stabilizer for the vaccine of from about 0.00 to about 99.9998 percent by weight.

14. The vaccine stabilizer set forth in claim 13, further including:
said thermal stabilizer being from about 0.00 to about 75.00 percent by weight.

15. The vaccine stabilizer set forth in claim 13, further including:
said thermal stabilizer being at least one chemical selected from the group consisting of proteins, poly-amino or -imine compounds and polyhydric alcohols.

16. The vaccine stabilizer set forth in claim 15, further including:
said thermal stabilizer being at least one chemical selected from the group consisting of sodium caseinate, calcium caseinate, isolated soy protein, serum albumin, egg albumin and hydrolyzed gelatin, lysine, arginine, cystine, diaminoethane, diaminopropane, diaminobutane, diaminopentane, spermidine, and poly(ethylenimine), and sorbitol, mannitol, and dulcitol.

17. The vaccine stabilizer set forth in claim 1, further including:
a sugar for an energy source for the vaccine of from about 0.00 to about 99.9998 percent by weight.

18. The vaccine stabilizer set forth in claim 17, further including:
said sugar being from about 0.00 to about 85.00 percent by weight.

19. The vaccine stabilizer set forth in claim 17, further including:
said sugar being at least one water soluble sugar selected from the group consisting of sucrose, glucose, dextrose, lactose, mannose and fructose.

20. The vaccine stabilizer set forth in claim 1, further including:
a diluent of from about 0.00 to about 99.9998 percent by weight.

21. The vaccine stabilizer set forth in claim 20, further including:
said diluent being water.

22. A vaccine stabilizer for adjusting water quality that adversely effects the life of a vaccine to be administered to livestock, consisting essentially of:
a reducing agent to neutralize oxidizing water sanitizers of at least about 0.0002 percent by weight;
a buffer to adjust pH of from about 0.00 to about 99.9998 percent by weight;
a thermal stabilizer for the vaccine of from about 0.00 to about 99.9998 percent by weight;
a coloring agent to provide a visual reference for aiding in determining the administration of the vaccine to the livestock of from about 0.00 to about 99.9998 percent by weight;
a sugar for an energy source for the vaccine of from about 0.00 to about 99.9998 percent by weight; and
diluent of from about 0.00 to about 99.9998 percent by weight.

23. The vaccine stabilizer set forth in claim 22, further including:
said reducing agent being from about 0.0002 to about 70.0 percent by weight.
said buffer being from about 0.00 to about 20.0 percent by weight;
said thermal stabilizer being from about 0.00 to about 75.0 percent by weight;
said coloring agent being from about 0.00 to about 3.5 percent by weight;
said sugar being from about 0.00 to about 85.0 percent by weight; and
said diluent being from about 0.00 to about 99.9998 percent by weight.

24. A vaccine stabilizer for adjusting water quality that adversely effects the life of a vaccine to be administered to livestock, consisting essentially of:
a reducing agent to neutralize oxidizing water sanitizers of at least about 0.0002 percent by weight, said reducing agent being at least one chemical selected from the group consisting essentially of sodium thiosulfate, sodium metabisulfite, sodium bisulfite, sodium sulfite, ammonium bisulfite and ammonium thiosulfate;
a buffer to adjust pH of from about 0.00 to about 99.9998 percent by weight,
said buffer being at least one chemical selected from the group consisting of phosphates, carboxylates and bicarbonates;
a thermal stabilizer for the vaccine of from about 0.00 to about 99.9998 percent by weight,
said thermal stabilizer being at least one chemical selected from the group consisting of proteins, poly-amino or -imine compounds and polyhydric alcohols;
a coloring agent to provide a visual reference for aiding in determining the administration of the vaccine to the livestock of from about 0.00 to about 99.9998 percent by weight,
said coloring agent being at least one chemical selected from the group consisting of a water soluble FD&C compliant food color;
a sugar for an energy source for the vaccine of from about 0.00 to about 99.9998 percent by weight,
said sugar being at least one water soluble sugar selected from the group consisting of sucrose, glucose, lactose, mannose and fructose; and
diluent of from about 0.00 to about 99.9998 percent by weight.

25. The vaccine stabilizer set forth in claim 24, further including:
said buffer being at least one chemical selected from the group consisting of sodium phosphate, potassium phosphate, sodium citrate, calcium lactate, sodium succinate, sodium glutamate, sodium bicarbonate, and potassium bicarbonate; and
said thermal stabilizer being at least one chemical selected from the group consisting of sodium caseinate, calcium caseinate, isolated soy protein, serum albumin, egg albumin and hydrolyzed gelatin, lysine, arginine, cystine, diaminoethane, diaminopropane, diaminobutane, diaminopentane, spermidine, and poly(ethylenimine), and sorbitol, mannitol, and dulcitol.

26. A method of treating livestock with a vaccine, comprising the steps of:
providing a quantity of water for treating livestock with the vaccine;
mixing a reducing agent selected to neutralize oxidizing water sanitizers in the water;
mixing the vaccine in the water after the reducing agent has neutralized the water sanitzers; and
treating the livestock with the vaccine and water mixture.

27. The method set forth in claim 26, further comprising the steps of:
transporting the quantity of water through a conduit; and
adding the reducing agent to the water while the water is being transported for mixing therewith.

28. The method set forth in claim 27, further comprising the step of:
adding the vaccine to the water while the water is being transported for mixing therewith at a location downstream of the location where the reducing agent is added.

29. The method set forth in claim 26, further comprising the step of:
adding a buffer to the water with the reducing agent to adjust the pH.

30. The method set forth in claim 26, further comprising the step of:
adding a coloring agent to the water with the reducing agent to provide a visual reference to aid in identifying the livestock receiving the vaccine.

31. A method of treating livestock with a vaccine, comprising the steps of:
transporting water to a location for treating livestock with a vaccine;
selecting a reducing agent that neutralizes oxidizing water sanitizers being transported in the water while not adversely effecting the vaccine;
adding the selected reducing agent to the water before reaching the location; and
mixing the vaccine in the water after the reducing agent is added to the water and before the water reaches the location.

32. The method set forth in claim 31, further comprising the step of:
adding a buffer to the water with the reducing agent to adjust the pH.

33. The method set forth in claim 31, further comprising the step of:
adding a coloring agent to the water with the reducing agent to provide a visual reference to aid in identifying the livestock receiving the vaccine.
